(19) United States
(12) Patent Application Publication
(10) Pub. No.: US 2024/0130942 A1
(43) Pub. Date: Apr. 25, 2024

(54) Composition for Treating Hair and Skin

(71) Applicant: DR. KURT WOLFF GMBH & CO. KG, Bielefeld (DE)

(72) Inventors: Erik SCHULZE ZUR WIESCHE, Bielefeld (DE); Maike BECKER, Bielefeld (DE); Jörn Michael VÖLKER, Bielefeld (DE)

(73) Assignee: DR. KURT WOLFF GMBH & CO. KG, Bielefeld (DE)

(21) Appl. No.: 18/247,527

(22) PCT Filed: Oct. 1, 2021
(86) PCT No.: PCT/EP2021/077176

§ 371 Date: Mar. 31, 2023

(30) Foreign Application Priority Data
Oct. 2, 2020 (DE) 10 2020 125 874.7

(51) Int. Cl. A61K 8/44; A61K 31/198; A61P 17/02; A61P 17/14; A61Q 7/00; A61Q 19/00

(52) U.S. Cl. CPC A61K 8/44; A61K 31/198; A61P 17/02; A61P 17/14; A61Q 7/00; A61Q 19/007; A61K 9/0014; A61K 2800/592

(57) ABSTRACT

The present invention relates to a topical composition comprising dimethylglycine and/or a salt of dimethylglycine and having a pH of 3.0 to 5.9. In addition, the present invention relates to the use (cosmetic and/or medicinal) of this composition for the treatment of hair and/or skin.

The present invention relates to a composition containing dimethylglycine and/or a salt of dimethylglycine. In addition, the present invention relates to the use (cosmetic and/or medicinal) of this composition for the treatment of hair and/or skin.

Human hair has largely lost its importance as a protective function for the body. However, healthy hair has great cultural significance for women and men worldwide and is often seen as a sign of wealth and health. Consequently, thinning hair caused by hair loss, for example, often negatively affects the quality of life. Hair loss caused by hormones such as androgens is known as androgenetic alopecia (alopecia androgenetica or AGA) and is the most common cause of hair loss, especially in men but also in women. A distinction can be made between male and female hair loss patterns.

Hair growth and hair regrowth depend on an adequate supply of nutrients to the hair follicles, which is provided by the blood. Reduced blood flow (microcirculation) in the scalp can therefore promote or even cause hair loss. For example, bald men have significantly less blood flow to the vertex region of the scalp than men with normal scalp hair. This significantly reduced microcirculation can therefore be an explanation for the hair loss and the lack of hair regrowth (transition from telogen to anagen), for example in androgenetic alopecia. Overall, hair loss can be seen as one of the first clinical signs of reduced blood flow in the peripheral vessels.

To date, there are few approved pharmaceutical treatments for hair loss such as androgenetic alopecia, and existing treatments are either not effective enough or come with unpleasant side effects. The situation is similarly unsatisfactory, for example, when it comes to treatment options for the acute onset of circular hair loss caused by inflammation, which is also known as alopecia areata or AA for short, and for hair loss caused by other causes.

In addition to the hair as a so-called skin appendage, the functionality of the skin is also of great importance for a healthy appearance. Physiological effects (such as inflammation) that affect the hair or hair roots also affect the skin in a similar way. Therefore, the treatment of the skin is of great importance both for medical reasons, such as an improved protective function and strengthened barrier properties, and for purely optical-aesthetic reasons, such as a smoother and simply more beautiful complexion.

There is therefore still a need for improved treatment methods, in particular a need for compositions which can be used topically to combat hair loss, with these compositions having no or only negligible side effects. In particular, there is a need for compositions which are pharmaceutical in nature and do not show any side effects or which are cosmetic in nature.

There is also a need for improved skin care compositions that improve both the feel and appearance of the skin and at the same time strengthen the skin's protective and barrier function.

On this basis, it was the object of the present invention to provide a well-tolerated composition for the treatment of hair and the skin, in particular for the prevention and/or treatment of hair loss. Furthermore, this composition should be able to be applied topically and overcome the disadvantages of the compositions known from the prior art.

Surprisingly, this object was achieved by the composition according to claim 1 and its use according to claim 8. Preferred embodiments are specified in the dependent claims.

According to the invention, the object is achieved by providing a composition which comprises dimethylglycine and/or a salt of dimethylglycine, the composition having a pH in the range from 3.0 to 5.9.

Surprisingly, it has been shown that the composition according to the invention with dimethylglycine and/or a salt of dimethylglycine has excellent efficacy in the treatment of hair loss, in particular hereditary and age-related hair loss, when the pH of the composition is in the range of 3.0 to 5.9. The composition activates the skin and significantly improves the supply of nutrients and oxygen to the skin and hair roots. In addition, it is medically and cosmetically extremely well tolerated on the skin.

Dimethylglycine (N,N-dimethylglycine) is found in plants, animals and humans, although it is only formed in very small amounts in humans. It is formed as an intermediate in a multistep biosynthesis of glycine from choline by transamination of betaine with betaine homocysteine methylase.

N,N-Dimethylglycine, also (dimethylamino)acetic acid, is represented by the following Chemical Formula 1:

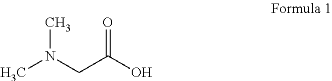

Not only the use of dimethylglycine, but also that of its salts, solvates and hydrates is inventive. These are preferably pharmaceutically or cosmetically acceptable salts of dimethylglycine. The salt is particularly preferably a water-soluble salt having a solubility in water of at least 10 g/l at 20° C.

In a preferred embodiment, the salt of dimethylglycine is an alkali, alkaline earth or ammonium salt of dimethylglycine.

Examples are sodium, potassium, calcium, magnesium and ammonium salts. In case of the ammonium salts, the ammonium cation carries one to four alkyl groups, each independently having 1 to 4 carbon atoms. Preferred are the sodium and potassium salts of dimethylglycine, especially the sodium salt of dimethylglycine, namely sodium N,N-dimethylglycinate.

In an alternatively preferred embodiment, the salt of dimethylglycine may be the salt of an inorganic and/or organic acid with dimethylglycine.

Examples of salts of dimethylglycine with an inorganic acid are the hydrochloride, hydrobromide, hydroiodide, hydrogen sulfate, sulfate, hydrogen sulfite, sulfite, hydrogen carbonate, carbonate, monophosphate, diphosphate and triphosphate of dimethylglycine, and mixtures thereof. Particularly preferred is the hydrochloride of dimethylglycine.

Examples of salts of dimethylglycine with an organic acid are the acetate, lactate, citrate, succinate, fumarate, maleate and benzoate of dimethylglycine, and mixtures thereof.

It is assumed that dimethylglycine and/or a salt of dimethylglycine according to the invention improve cell activity and oxygen turnover in the keratinocytes and thus also promote cell activity in the (scalp) skin and in the hair follicles. Furthermore, the skin is smoothed, the skin barrier is strengthened and wound healing is supported. According to the invention, it thus achieves a significant hair root and skin strengthening effect, in particular in the treatment of everyday or age-related stressed or weakened skin and hair loss, such as hereditary and age-related hair loss.

According to the invention, the pH of the topical composition is 3.0 to 5.9, preferably 3.5 to 5.4 and particularly preferably 4.0 to 5.0 (measured at 21° C. using a pH meter, Mettler-Toledo SevenCompact S220). In this range, the compositions according to the invention are not only stable chemically, physically and microbiologically particularly, but also extremely well tolerated medically and cosmetically by the skin and hair. Particularly suitable pH values for the composition according to the invention are: 3.5; 3.6; 3.7; 3.8; 3.9; 4.0; 4.1; 4.2; 4.3; 4.4; 4.5; 4.6; 4.7; 4.8; 4.9; 5.0; 5.1; 5.2; 5.3; and 5.4. The pH of the composition is preferably adjusted using one or more pH modifiers. Suitable pH modifiers can be acids, bases and/or buffer systems to stabilize or affect the pH of the composition. Typical pH modifiers according to the present invention are adipic, citric, malic, succinic, tartaric, ascorbic, phosphoric, lactic and fumaric acids and their corresponding salts, as well as sodium alginate, polyacrylic acid, sodium carbonate and sodium bicarbonate. In the context of pH modifiers, the term salt refers to alkali metal salts or alkaline earth metal salts unless otherwise specified.

The composition according to the invention preferably contains dimethylglycine and/or a salt of dimethylglycine in a proportion of 0.00001 to 25.0% by weight, based on the total weight of the composition. In a preferred embodiment, the composition according to the invention contains dimethylglycine and/or a salt of dimethylglycine in an amount of 0.001% to 10.0% by weight, more preferably from 0.01% to 8.0% by weight, more preferably from 0.1% to 6.0% by weight, even more preferably from 0.3% to 5.0% by weight, in particular from 0.5% to 3.0% by weight, based on the total weight of the composition. In a preferred embodiment according to the invention, the composition according to the invention can contain 0.1% by weight, 0.2% by weight, 0.3% by weight, 0.4% by weight, 0.5% by weight, 0.6% by weight %, 0.7% by weight, 0.8% by weight, 0.9% by weight, 1.0% by weight, 1.1% by weight, 1.2% by weight, 1.3% by weight, 1.4% by weight, 1.5% by weight, 2.0% by weight or 2.5% by weight dimethylglycine and/or a salt of dimethylglycine, in each case based on the total weight of the composition. The composition according to the invention preferably contains dimethylglycine and/or a salt of dimethylglycine as a pure chemical substance, including the respective solvates and hydrates (e.g., the dihydrate of sodium dimethylglycinate), since this increases the purity of the composition and reduces the occurrence of undesirable side effects. For this reason, the composition according to the invention preferably contains the chemical derivatives of dimethylglycine, selected from methylglycine, trimethylglycine, (2-hydroxyethyl)-trimethylammonium, and trimethyl-hydroxybutyrobetaine, in concentrations of less than 0.01% by weight, based on the total weight of the Composition. The compositions according to the invention are particularly preferably completely free of these derivatives. Preferably, dimethylglycine and/or a salt of dimethylglycine is dissolved homogeneously in a continuous or discontinuous aqueous phase, particularly preferably in a continuous aqueous phase.

In a preferred embodiment, the composition according to the invention contains at least one other active ingredient, wherein the at least one other active ingredient is selected from caffeine, menthol, biotin, zinc PCA, niacinamide, panthenol, ectoine, ubiquinone-10, taurine, pantolactone, echinacea, tocopheryl acetate and combinations thereof. The combination of dimethylglycine or the salts of dimethylglycine with caffeine is particularly preferred according to the invention. The active ingredient or combination of active ingredients preferably does not include the compounds methylmethionine, glycerophosphocholine, phosphatidylcholine, N-acylethanolamine and/or N-acylphosphatidylethanolamine, for example with a C1-C22 acyl radical.

The IUPAC name of caffeine is 1,3,7-trimethyl-3,7-dihydro-1H-purine-2,6-dione. Alternatively, caffeine is also referred to as 1,3,7-trimethylxanthine. Caffeine is represented by Chemical Formula 2 below.

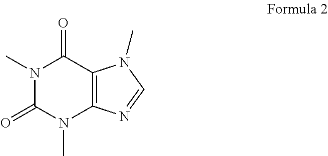

Caffeine is a methylxanthine alkaloid belonging to the methylxanthine class. It is a bitter, crystalline substance, can be considered a purine derivative, and is chemically related to the adenine and guanine bases of deoxyribonucleic acids and ribonucleic acid.

The active ingredient combination of dimethylglycine or a salt of dimethylglycine with caffeine according to the invention shows improved efficacy in the treatment of hair loss, in particular hereditary and age-related hair loss, than caffeine or dimethylglycine and/or a salt of dimethylglycine alone. The combination of active ingredients increases the microcirculation in the scalp to an unexpected extent and significantly improves the supply of nutrients and oxygen to the hair roots.

In a preferred embodiment, the composition according to the invention contains caffeine in a proportion of 0.001% to 3.0% by weight, more preferably 0.005% to 2.50% by weight, even more preferably 0.01% to 2.0% by weight, in particular 0.1% to 1.5% by weight, in each case based on the total weight of the composition. In a preferred embodiment according to the invention, the composition according to the invention can contain 0.01% by weight, 0.05% by weight, 0.1% by weight, 0.2% by weight, 0.3% by weight, 0.4% by weight, 0.5% by weight, 0.6% by weight, 0.7% by weight, 0.8% by weight, 0.9% by weight, 1.0% by weight, 1.1% by weight, 1.2% by weight, 1.3% by weight, 1.4% by weight, 1.5% by weight of caffeine, in each case based on the total weight of the composition.

The weight ratio between dimethylglycine and/or a salt of dimethylglycine and caffeine is preferably in a range from 10:1 to 1:10, preferably from 6:1 to 1:6, more preferably from 4:1 to 1:4, particularly preferably from 2:1 to 1:2. In a preferred embodiment according to the invention the weight ratio between dimethylglycine and/or a salt of dimethylglycine and caffeine is 0.5; 0.6; 0.7; 0.8; 0.9; 1.0; 1.1; 1.2; 1.3; 1.4; 1.5; 1.6; 1.7; 1.8; 1.9 or 2.0.

Also preferred according to the invention is the combination of dimethylglycine or one of the salts of dimethylglycine, either alone or particularly preferably together with caffeine, and one or more of the following active ingredients.

Alternatively, the topical composition of the present invention just does not contain caffeine. This embodiment is also referred to as an (completely) caffeine-free topical composition or a topical composition without caffeine according to the invention, to which, apart from that, all other preferred features according to the invention described herein apply.

Menthol is a monocyclic monoterpene alcohol and can be added to the composition according to the invention as an active ingredient which stimulates blood flow. In addition, menthol can cause a refreshing sensory stimulation of the scalp.

Biotin, which is also known as vitamin B₇ or vitamin H, is a water-soluble vitamin from the B complex. According to the invention, biotin can further reduce hair loss and strengthen the skin.

Zinc PCA is the zinc salt of L-pyrrolidone carboxylate and can be added to the composition according to the invention as a substance with an antimicrobial effect.

Niacinamide (also nicotinamide) is the amide of nicotinic acid and is also known as vitamin B₃. In addition to other properties such as a reduction in oxidative stress, the niacinamide according to the invention has a hair growth-stimulating effect.

Panthenol is a provitamin which is converted to pantothenic acid (vitamin B₅) in the body. The latter is part of coenzyme A and therefore important for skin metabolism. According to the invention, the skin's elasticity and moisture are further improved by the action of panthenol. In addition, itching and inflammation are relieved and wound healing is promoted.

Ectoin is a cyclic amino acid and is present in aqueous solution as a resonance-stabilized zwitterion. Ectoin has a moisturizing effect and, according to the invention, further stabilizes the natural structure of hair. It has also been shown that ectoine protects against UV radiation and can be helpful in the treatment of inflammatory diseases.

Ubiquinone-10 (Q10 or Coenzyme Q₁₀) is a quinone derivative. The Q10 belonging to the ubiquinone pool is considered an antioxidant and, according to the invention, has a stabilizing effect on the skin and hair and in particular the hair roots.

According to the invention, taurine or 2-aminoethanesulfonic acid, as an antioxidant, also has a further stabilizing effect on the skin and hair and in particular the hair roots.

Pantolactone comes from the group of substituted lactones and, according to the invention, further stimulates the growth factors of the hair roots.

According to the invention, echinacea has a calming effect on the skin and scalp and relieves itching and tension that occurs. In addition, echinacea can stimulate blood circulation in the scalp and thus supply the hair follicles with blood rich in oxygen and nutrients, which has a further stabilizing effect on the hair and in particular the hair roots.

Tocopheryl acetate exhibits antioxidant properties and, according to the invention, has a further stabilizing effect on the skin and the hair and in particular the hair roots.

In a preferred embodiment according to the invention, the composition according to the invention contains at least one other active substance selected from menthol, biotin, zinc PCA, niacinamide, panthenol, ectoine, ubiquinone, taurine, pantolactone, echinacea, tocopheryl acetate and combinations thereof, each in a proportion of 0.001% to 10.0% by weight, more preferably from 0.005% to 7.50% by weight, even more preferably from 0.01% to 5.0% by weight, in particular 0.1% to 3.0% by weight, based on the total weight of the composition.

In addition, the composition according to the invention can be water-based. This means that it contains, for example, 45.0% to 85.0% by weight of water.

The composition preferably has a viscosity of 800 to 6000 mPa·s, particularly preferably from 1000 to 5700 mPa·s and very particularly preferably from 2500 to 5500 mPa·s, measured in each case in accordance with DIN 53019-1:2008-09 using a rheometer Haake RheoStress1 (ThermoFisher Scientific) at 20° C. and a shear rate of 10/s in plate-plate geometry (rotary body PP60 Ti).

In one embodiment, the composition includes a surfactant. The surfactant can be an anionic, nonionic, cationic or zwitterionic surfactant. The surfactant is preferably an anionic or nonionic surfactant, in particular an anionic or nonionic surfactant which is as mild as possible (i.e., particularly kind to the skin). According to the invention, nonionic surfactants are used in particular because of their very good emulsification properties and their excellent skin care properties. Anionic surfactants are preferred because they have particularly high cleaning performance. They are therefore particularly useful in cleaning compositions such as shampoos. Cationic surfactants have excellent hair care properties and are used according to the invention in particular in hair care compositions such as in conditioners, shampoos and treatments.

The composition according to the invention contains surfactants preferably in an amount of 2% to 40% by weight, in particular 5% to 30% by weight, preferably 7% to 20% by weight, particularly preferably 10% to 17% by weight, based in each case on the total weight the composition. Suitable amounts of surfactant are: 8% by weight; 9% by weight; 10% by weight; 11% by weight; 12% by weight; 13% by weight; 14% by weight; 15% by weight; 16% by weight; 17% by weight; 18% by weight; 19% by weight; 20% by weight; 21% by weight; 22% by weight; 23% by weight; 24% by weight; 25% by weight, in each case based on the total weight of the composition. The topical composition according to the invention particularly preferably contains one or more anionic surfactants in an amount of 0.1% to 20% by weight, preferably from 1% to 17% by weight and particularly preferably from 5 to 15% by weight, in each case based on the total weight the composition. Suitable amounts of anionic surfactant are: 1% by weight; 2% by weight; 3% by weight; 4% by weight; 5% by weight; 6% by weight; 7% by weight; 8% by weight; 9% by weight; 10% by weight; 11% by weight; 12% by weight; 13% by weight; 14% by weight; 15% by weight; 16% by weight; 17% by weight; 18% by weight; 19% by weight; 20% by weight, in each case based on the total weight of the composition. In these amounts, the surfactants have a particularly high cleaning performance and are extremely well tolerated by the skin, scalp and hair.

The surfactants of the present invention are described, inter alia, in the book “Surfactants and interfacial phenomena”, by Milton Rosen and Joy Kunjappu, John Wiley & Sons, Inc., Publisher, 2012, 4th edition.

In a preferred embodiment, the surfactant is an anionic surfactant selected from alkyl sulfonates, alkyl sulfates, alkyl ether sulfates, alkyl phosphates, alkyl sarcosinates, alkyl taurates, amino acid surfactants, and mixtures thereof. More preferably, the surfactant is selected from alkyl sulfates, alkyl sarcosinates, alkyl taurates, alkyl glutamate such as sodium cocoyl glutamate/disodium cocoyl glutamate, alkyl glycinate, alkyl alaninate such as sodium cocoyl alaninate, and mixtures thereof. Also preferred because of their cleaning performance are fatty alcohol polyglycerol ether sulfates, monoglyceride sulfates, mono- and/or dialkyl sulfosuccinates, fatty acid isethionates, and α-olefin sulfonates.

Alkyl sulfates have the generic formula ROSO₃M, alkyl sarcosinates have the generic formula RC(O)N(CH₃)CH₂CO₂M, and alkyl taurates have the generic formula RC(O)N(CH₃)CH₂CH₂SO₃M, where each R is C₄-C₂₆ alkyl or C₄-C₂₆ alkenyl and M is a water soluble cation such as ammonium, sodium or potassium. Preferably, M is a sodium cation. Preferably, R is a C₁₂-C₁₆ alkyl or a C₁₂-C₁₈ alkyl.

In one embodiment, the surfactant is a nonionic surfactant (also referred to as a nonionic emulsifier). Non-limiting examples include glycerol fatty acid esters, polyoxyethylene ethers of one or more fatty alcohols, alkoxylated fatty acid alkyl esters, polyglycerol ethers of fatty alcohols, polyglycerol esters of fatty acids, polyethylene glycol and/or polypropylene glycol ethers, fatty acid amides, alkyl phenol polyglycol ethers, amine oxides, and alkyl polyglucosides.

In one embodiment, the surfactant is selected from the group of glycerol fatty acid esters, polyoxyethylene ethers of one or more fatty alcohols, polyglycerol ethers of fatty alcohols, polyglycerol esters of fatty acids, and mixtures thereof.

In the present invention, the term “glycerol fatty acid ester” refers to a glycerol mono- or glycerol di-fatty acid ester. Glycerol di-fatty acid esters have the formula R³—COO—(CH₂CH(OH)CH₂)—OOR⁴ or R³—COO—(CH₂CH(OOR⁴)CH₂)—OH. Glycerol monofatty acid esters have the formula R³—COO—(CH₂CH(OH)CH₂)—OH or HO—(CH₂CH(OOR³)CH₂)—OH. Here, R³ and R⁴ are independently selected from C₆-C₂₈ alkyl and C₆-C₂₈ alkenyl. Glycerol mono-fatty acid esters contain a glycerol group attached to a single fatty acid via an ester linkage. Examples are glycerol monostearate, glycerol monobehenate, glycerol monocaprylate, glycerol monocaprate and glycerol monolaurate.

Polyoxyethylene ethers are compounds of the formula R⁵(OC₂H₃)ₙOH, wherein R⁵ is selected from C₆-C₂₈ alkyl, C₆-C₂₈ alkenyl, substituted and unsubstituted phenoxy groups; and n is an integer greater than 1. The polyoxyethylene ether of one or more fatty alcohols is preferably selected from the group consisting of steareth-2, steareth-21, macrogol cetostearyl ether-12, ceteareth-25, macrogol cetostearyl ether-20, and mixtures of the aforementioned compounds. More preferably, the polyoxyethylene ether is a compound selected from the group consisting of ceteareth-25, macrogol cetostearyl ether-20, and mixtures of the aforementioned compounds.

The term “polyglycerol ethers of fatty alcohols” refers to a compound of the formula R⁶O—(C₃H₆O₂)ₙ—H, wherein R⁶ is a branched or linear C₆-C₂₈ alkyl or C₆-C₂₈ alkenyl and n is an integer greater than 1, preferably an integer from 2 to 10. It is preferred that the composition contains 0.01% to 15.0% by weight, 0.1% to 10.0% by weight, or 1% to 5.0% by weight polyglycerol ether.

The term “polyglycerol esters of fatty acids” refers to compounds containing both a polyglycerol moiety and at least one C₆-C₂₆ alkyl or C₆-C₂₆ alkenyl carboxylic acid moiety. These compounds may have the formula R⁷—R⁸—(C₃H₆O₂)ₙ—H, wherein R⁷ is a C₆-C₂₆ alkanoate or C₆-C₂₆ alkenoate radical and R⁸ is a suitable connecting molecule or a direct bond. Thus, the polyglycerol moiety and the C₆-C₂₆ alkyl or C₆-C₂₆ alkenyl carboxylic acid moiety may be linked directly through an ester linkage or may contain a linking moiety linking these two moieties together. Non-limiting examples of this group are polyglyceryl 3-methylglucose distearate, polyglycerol polycrinoleate, polyglyceryl dimerate isostearate, polyglyceryl 2-laurate, polyglyceryl 2-sesquiisostearate, polyglyceryl 3-distearate (Cremophor GS 32), polyglyceryl 3-oleate, polyglyceryl 3-methyl glucose distearate, polyglyceryl 4-caprate (polyglycerol caprate T2010190), polyglyceryl 4-diisostearate/polyhydroxystearate/sebacate (Isolan GPS), and polyglyceryl 4-isostearate.

In one embodiment, the surfactant comprises a cationic surfactant, such as a quaternary surfactant. Quaternary surfactants contain at least one N atom which is covalently linked to 4 alkyl or aryl groups. Regardless of the pH value, this leads to a positive charge. Alkyl betaine, alkyl amidopropyl betaine and alkyl amidopropyl hydroxysulfain are advantageous. The cationic surfactants used according to the invention can also preferably be selected from the group of quaternary ammonium compounds, in particular benzyltrialkylammonium chlorides or bromides, such as benzyldimethylstearylammonium chloride, also alkyltrialkylammonium salts, for example cetyltrimethylammonium chloride or bromide, alkyldimethylhydroxyethylammonium chlorides or bromides, dialkyldimethylammonium chlorides or bromides, alkylamidethyltrimethylammonium ether sulfates, alkylpyridinium salts, for example lauryl or cetylpyrimidinium chloride, imidazoline derivatives and compounds with a cationic character such as amine oxides, for example alkyldimethylamine oxides or alkylaminoethyldimethylamine oxides. It is particularly advantageous to use cetyltrimethylammonium salts.

In a further embodiment, the composition according to the invention contains at least one additive. Additives which are customarily used in shampoos, conditioners and emulsions for treating the skin, scalp and hair are preferred. The at least one additive can be present in a proportion of 0.01% to 12.0% by weight, more preferably from 0.25% to 10.0% by weight, in particular from 1.0% to 7.0% by weight.

The at least one additive can also be selected from the group consisting of hair conditioners, lipid replenishers, preservatives, stabilizers, fragrances, antioxidants, rheology modifiers, thickeners, conditioning agents, dyes, pearlescent agents, lightening agents, solvents, and combinations thereof.

Hair conditioners can reduce static charges in hair by neutralizing electrical charge on their surface. Examples of hair conditioning agents are quaternary ammonium compounds.

Lipid replenishers, also referred to as lipid replenishing agents or superfatting agents, are lipophilic substances that can prevent a disruptive effect on the epidermal barrier function. Examples of lipid replenishers are wool wax, squalene, liquid paraffin, vegetable oils, silicones and cetyl palmitate.

Preservatives are substances used to preserve the composition by killing and/or inhibiting the growth of microorganisms that degrade the composition. Preferably, the preservatives can be selected from the group consisting of benzoic acid, benzoic acid derivatives, sorbic acid, sorbic acid derivatives, salicylic acid, salicylic acid derivatives, phenoxyethanol, parabens, and combinations thereof. In a preferred embodiment, sodium benzoate and/or potassium sorbate are utilized as preservatives in the composition according to the invention. Sodium benzoate releases benzoic acid and potassium sorbate sorbic acid in a slightly acidic environment. An antimicrobial effect is ascribed to both acids.

Stabilizers can protect photosensitive components from radiation and are preferably UV absorbers such as benzophenone derivatives.

The addition of fragrances can provide a pleasant odor to the composition. Examples are the perfumes known to those skilled in the art.

An antioxidant or anti-oxidant is a chemical compound that retards or completely eliminates oxidation of other components in the composition according to the invention. Examples of possible antioxidants are citric acid, ascorbic acid and butylated hydroxyanisole.

Rheology modifiers and thickeners can help to improve the application properties of the composition according to the invention. The addition of table salt (sodium chloride) can be considered as a rheology modifier and thickener. The flowability of the composition according to the invention can be influenced within certain limits and adjusted to the required level by adding sodium chloride. Naturally occurring gel formers, which are preferably selected from agar, xanthan gum, cellulose and/or cellulose derivatives or alginic acid, can also be used as thickeners.

In the context of the application, care products should be understood to mean substances which care for the hair and/or the scalp. Hydrolyzed wheat protein and allantoin nourish the scalp and hair. Hydrolyzed wheat protein mainly has moisturizing properties.

Dyes are optionally used to give the composition according to the invention a characteristic color so that it can be easily distinguished from other products. In the context of the invention, however, dyes can also be used to color human hair.

A solvent or solvent mixture which is customary for a person skilled in the art in this field can be used as the solvent. Preferred solvents are ethanol or butylene glycol, especially 1,4-butylene glycol, propylene glycol and isopropyl alcohol. Ethanol is preferred. These solvents can preferably be contained in the composition according to the invention in an amount of 0.1% to 70% by weight. They are very particularly preferably present in an amount of 0.1% to less than 5.0% by weight. Solvents can preferably be contained in compositions that remain on the head when they are used (leave-on formulations). Their use can lead to a feeling of freshness and, due to the rapid drying, has a very low impact on the hairstyle. In addition, solvents help the penetration of the active ingredients and thus increase their efficacy.

According to the invention, the composition is applied topically. A topical application means an external application, in particular a local, external application. Compositions according to the invention are preferably skin, scalp and hair care products (i.e., treatment agents such as shampoos, conditioners, treatments, emulsions, lotions, shower gels, day creams, face creams, face fluids and/or a tonic) and not (pure) styling products. In other words, the topical compositions according to the invention just do not have the primary purpose of hair styling, where the effect is exerted on the hair itself and not on the scalp. Rather, the focus is on the medical/pharmaceutical or cosmetic treatment of the skin, scalp and hair.

In a preferred embodiment, the components, in particular the active ingredient dimethylglycine or the salts of dimethylglycine and the other active ingredients for treating the skin and hair, if present, are homogeneously distributed/dissolved in a continuous or discontinuous aqueous phase in the composition according to the invention, preferably in a continuous aqueous phase. The term “aqueous phase” includes the possible presence of water-miscible organic solvents (e.g., alcohols). This means in particular that the composition according to the invention preferably has no further carrier substances. The composition according to the invention preferably contains no lamellar structure (in particular no vesicles and/or lamellar double-membrane structure and/or liposomes). It preferably also contains no substance(s) forming lamellar structures (in particular vesicles and/or lamellar double membrane structures).

In a preferred embodiment, the composition according to the invention can be in the form of a leave-on formulation or rinse-off formulation.

A leave-on formulation is characterized in that it remains in contact with the (scalp) skin to be treated and/or the hair to be treated after application. This creates a kind of active ingredient depot that can develop its efficacy over a longer period of time. Leave-on formulations can be, for example, in the form of a hydrogel or emulsion or an aqueous or aqueous-alcoholic solution (tonic). Leave-on formulations are preferably in the form of slightly viscous formulations, so that they remain in concentrated form on the scalp and/or the hair and are less able to spread into the hair shaft. However, with these leave-on formulations it is important to ensure that the hair is not stressed by the substances that give it consistency, as they then leave a somewhat unkempt impression.

A rinse-off formulation is characterized in that the applied components of the composition according to the invention are rinsed out after application. In this way, excessive stress on the scalp and/or hair can be avoided. Rinse-off formulations can preferably be in the form of a shampoo, conditioner or a (head) skin and/or hair treatment. In a preferred embodiment, an exposure time of about 2-5 minutes should be provided for a composition according to the invention that is applied as a rinse-off formulation. In this way, the necessary penetration of the active ingredients into the scalp and/or into the hair roots can be made possible.

The present invention further relates to the use of the composition according to the invention for treating hair, scalp and/or skin.

In a preferred embodiment, the treatment of hair and scalp relates to the treatment and/or prevention of hair loss. The term hair loss includes alopecia areata (round hair loss), alopecia androgenetica (hereditary hair loss) in women and men, diffuse alopecia (diffuse hair loss), age-related hair loss (senescent alopecia) and hair loss caused by chemotherapy.

The hair loss to be treated is particularly preferably hereditary hair loss (alopecia androgenetica).

The hair loss to be treated is also particularly preferably age-related hair loss (senescent alopecia).

In a preferred embodiment, the treatment of the skin concerns the purely optical-aesthetic improvement, such as the creation of a smoother and simply more beautiful complexion. However, cosmetic/medical effects that go beyond this are also achieved according to the invention, such as an improved protective function and a strengthened barrier property. In addition, wound healing is improved.

In other words, the use according to the invention leads to a significant improvement in the epidermal barrier functions and the epidermal barrier integrity, to an improvement in the appearance of the skin and to an increase in the moisture content of the skin. This goes hand in hand with an increase in the cohesion of the stratum corneum and the homeostasis of the skin barrier and ultimately results in improved protection against infections (microbial diseases).

In the case of medical use, the invention encompasses both the therapeutic and the prophylactic treatment of skin diseases. The diseases are preferably microbial skin infections, skin inflammation, rough skin, dry skin, skin irritation, itching, pruritus, allergies, psoriasis, psoriatic arthritis, eczema, scleroderma, atopic dermatitis, contact dermatitis, systemic lupus erythematosus, acne and susceptibility to contact allergies.

The non-therapeutic (i.e., purely cosmetic) use includes in particular the treatment of cosmetic indications of the skin, selected in particular from rough skin, dry skin, skin irritations, itching and pruritus, as well as the prevention of skin infections and the reduction of susceptibility to contact allergies.

The invention shall be illustrated on the basis of the compositions below, without however wishing to limit it to the specific examples.

### EXPERIMENTAL SECTION

The following compositions were prepared by homogenization procedures known to those skilled in the art. The amount of the components was chosen in each case so that their proportion by weight corresponds to the proportions by weight indicated in the finished composition.

### Example 1

Composition in the form of a shampoo (pH 4.8), which contains the following components:

| | |
|---|---|
| Sodium myreth sulfate | 4.0% by weight |
| Sodium laureth sulfate | 4.0% by weight |
| Disodium laureth sulfosuccinate | 3.0% by weight |
| Tocopheryl acetate | 0.3% by weight |
| Citric acid | 0.2% by weight |
| Caffeine | 1.0% by weight |
| Na-dimethylglycinate | 1.0% by weight |
| Perfume | 0.3% by weight |
| Potassium sorbate | 0.2% by weight |
| Sodium benzoate | 0.1% by weight |
| Water | q.s. to 100% by weight |

### Example 1a

Composition in the form of a shampoo (pH 4.9), which contains the following components:

| | |
|---|---|
| Sodium myreth sulfate | 4.0% by weight |
| Sodium laureth sulfate | 4.0% by weight |
| Disodium laureth sulfosuccinate | 3.0% by weight |
| Tocopheryl acetate | 0.3% by weight |
| Citric acid | 0.2% by weight |
| Na-dimethylglycinate | 1.0% by weight |
| Perfume | 0.3% by weight |
| Potassium sorbate | 0.2% by weight |
| Sodium benzoate | 0.1% by weight |
| Water | q.s. to 100% by weight |

### Example 2

Composition in the form of a shampoo (pH 5.3), which contains the following components:

| | |
|---|---|
| Sodium myreth sulfate | 4.0% by weight |
| Sodium laureth sulfate | 4.0% by weight |
| Disodium laureth sulfosuccinate | 3.0% by weight |
| Tocopheryl acetate | 0.3% by weight |
| Citric acid | 0.2% by weight |
| Dimethylglycine HCl | 1.0% by weight |
| Perfume | 0.3% by weight |
| Potassium sorbate | 0.2% by weight |
| Sodium benzoate | 0.1% by weight |
| Water | q.s. to 100% by weight |

### Example 3

Composition in the form of a shampoo (pH 4.4), which contains the following components:

| | |
|---|---|
| Sodium laureth-11 carboxylate | 1.0% by weight |
| Polyquaternium-4/hydroxypropyl starch | 2.0% by weight |
| copolymer | |
| Steareth-8 | 5.0% by weight |
| Cetyl alcohol | 2.0% by weight |
| Perfume | 0.5% by weight |
| Na-dimethylglycinate | 0.4% by weight |
| Hydrolyzed creatine | 0.5% by weight |
| Citric acid | 0.2% by weight |
| Sodium benzoate | 0.05% by weight |
| Potassium sorbate | 0.15% by weight |
| Water | q.s. to 100% by weight |

### Example 4

Composition in the form of an emulsion (pH 5.5) containing the following components:

| | |
|---|---|
| PEG-40 (hydrogenated castor oil) | 6.0% by weight |
| Cetearyl alcohol | 0.3% by weight |
| Cera Alba | 0.7% by weight |
| Myristyl myristate | 1.0% by weight |
| Hexyldecanol | 5.0% by weight |
| Dicapryl ether | 4.0% by weight |
| Dioctylcyclohexane | 3.0% by weight |
| Tocopheryl acetate | 1.0% by weight |
| Octyl methoxycinnamate | 2.0% by weight |
| 4-Isobutyl-dibenzoylmethane | 1.0% by weight |
| Ascorbyl palmitate | 0.2% by weight |
| Retinyl palmitate | 0.05% by weight |
| 1,4-Butylene glycol | 4.0% by weight |
| Xanthan gum | 0.3% by weight |
| Hexyldecanol & hexyldecyllaurate | 1.0% by weight |
| Citric acid | (pH 5.5) q.s. |
| Na-dimethylglycinate | 0.4% by weight |
| Perfume | 0.2% by weight |
| Water | q.s. to 100% by weight |

### Example 5

Composition in the form of an emulsion (pH 4.0) containing the following components:

| | |
|---|---|
| Glycerol monostearate | 2% by weight |
| Cetyl alcohol | 3% by weight |
| Paraffin oil, subliquidum | 15% by weight |
| Vaseline | 3% by weight |
| Caprylic acid triglyceride | 4% by weight |
| Octyldodecanol | 2% by weight |
| Hydrogenated coconut fat | 2% by weight |
| Glycerol | 3% by weight |
| Glycolic acid | (pH 4.0) q.s. |
| Na-dimethylglycinate | 0.4% by weight |
| Perfume oil | q.s. |
| Preservatives | q.s. |
| Water | q.s. to 100% by weight |

### Example 6

Composition in the form of an emulsion (pH 5.9) containing the following components:

| | |
|---|---|
| PEG-7 hydrogenated castor oil | 4% by weight |
| Wool wax alcohol | 1.5% by weight |
| Beeswax | 3% by weight |
| Triglyceride, liquid | 5% by weight |
| Vaseline | 9% by weight |
| Stearyl alcohol | 4% by weight |
| Paraffin oil, subliquidum | 4% by weight |
| Glycerol | 2% by weight |
| Magnesium sulfate 7 H2O | 0.7% by weight |
| Na-dimethylglycinate | 0.4% by weight |
| Lactic acid | (pH 5.9) q.s. |
| Perfume oil | q.s. |
| Preservatives | q.s. |
| Water | q.s. to 100% by weight |

### Example 7

Composition in the form of a shampoo (pH 5.0), which contains the following components:

| | |
|---|---|
| Sodium laureth sulfate | 12.0% by weight |
| Cocoamidopropyl betaine | 3.5% by weight |
| Disodium cocoamphodiacetate | 3.0% by weight |
| Citric acid | 0.2% by weight |
| Na-dimethylglycinate | 1.0% by weight |
| Perfume | 0.3% by weight |
| Potassium sorbate | 0.2% by weight |
| Sodium benzoate | 0.1% by weight |
| Water | q.s. to 100% by weight |

### Reference Example 1

Composition in the form of a shampoo (without sodium dimethylglycinate; pH 5.1) containing the following components:

| | |
|---|---|
| Sodium myreth sulfate | 4.0% by weight |
| Sodium laureth sulfate | 4.0% by weight |
| Disodium laureth sulfosuccinate | 3.0% by weight |
| Tocopheryl acetate | 0.3% by weight |
| Citric acid | 0.2% by weight |
| Perfume | 0.3% by weight |
| Potassium sorbate | 0.2% by weight |
| Sodium benzoate | 0.1% by weight |
| Water | q.s. to 100% by weight |

### Reference Example 2

Composition in the form of an oil-in-water emulsion (pH 4.0) containing the following components:

| | |
|---|---|
| Glycerol monostearate | 2% by weight |
| Cetyl alcohol | 3% by weight |
| Paraffin oil, subliquidum | 15% by weight |
| Vaseline | 3% by weight |
| Caprylic acid triglyceride | 4% by weight |
| Octyldodecanol | 2% by weight |
| Hydrogenated coconut fat | 2% by weight |
| Glycerol | 3% by weight |
| Glycolic acid | (pH 4.0) q.s. |
| Perfume oil | q.s. |
| Preservatives | q.s. |
| Water | q.s. to 100% by weight |

### Reference Example 3

Composition in the form of an oil-in-water emulsion (pH 8.0) containing the following components:

| | |
|---|---|
| Glycerol monostearate | 2% by weight |
| Cetyl alcohol | 3% by weight |
| Paraffin oil, subliquidum | 15% by weight |
| Vaseline | 3% by weight |
| Caprylic acid triglyceride | 4% by weight |
| Octyldodecanol | 2% by weight |
| Hydrogenated coconut fat | 2% by weight |
| Glycerol | 3% by weight |
| Sodium hydroxide | (pH 8.0) q.s. |
| Na-dimethylglycinate | 0.4% by weight |
| Perfume oil | q.s. |
| Preservatives | q.s. |
| Water | q.s. to 100% by weight |

Study Design and Results

1. As part of an in vitro study, the effect of Na-dimethylglycinate was examined in a pH range from less than pH 6 and greater than pH 3 in a cell culture model with human horn-forming keratinocyte cells. For this purpose, HaCaT cells were cultured for 1, 3, 5, and 7 days in DMEM medium (including fetal calf serum and an antibiotic-antimycotic mix) and, among other things, the viability, proliferation and migration of the cells were determined using suitable measurement methods, and the expression of the growth factors relevant for cell growth was measured.

Proof of Viability:

For this measurement, a so-called MTT assay was used to determine cellular metabolic activity as an indicator of cell viability and cytotoxicity. This colorimetric assay is based on the reduction of a yellow tetrazolium salt (3-(4,5-dimethylthiazol-2-yl)-2,5-diphenyltetrazolium bromide or MTT) to purple formazan crystals by metabolically active cells.

Human epidermal keratinocyte cells (HaCaT) were seeded in a 96-well plate at a cell density of 5,000 cells/well and cultured in medium (DMEM with 10% FBS, 1% penicillin-streptomycin, 0.5% fungizone). The next day and after a further 24 h as well as 48 h and 72 h of cultivation at 37° C. and 5% by volume CO₂, the cell culture medium without (control) or with the active ingredient concentrations of sodium dimethylglycinate (DMG) already present was changed The measurement was carried out analogously to previously published studies in B. I. Tóth, N. Dobrosi, A. Dajnoki, G. Czifra, A. Oláh, A. G. Szöllősi, I. Juhász, K. Sugawara, R. Paus, T. Bíró, J Invest Dermatol 2011, 131, 1095-1104.

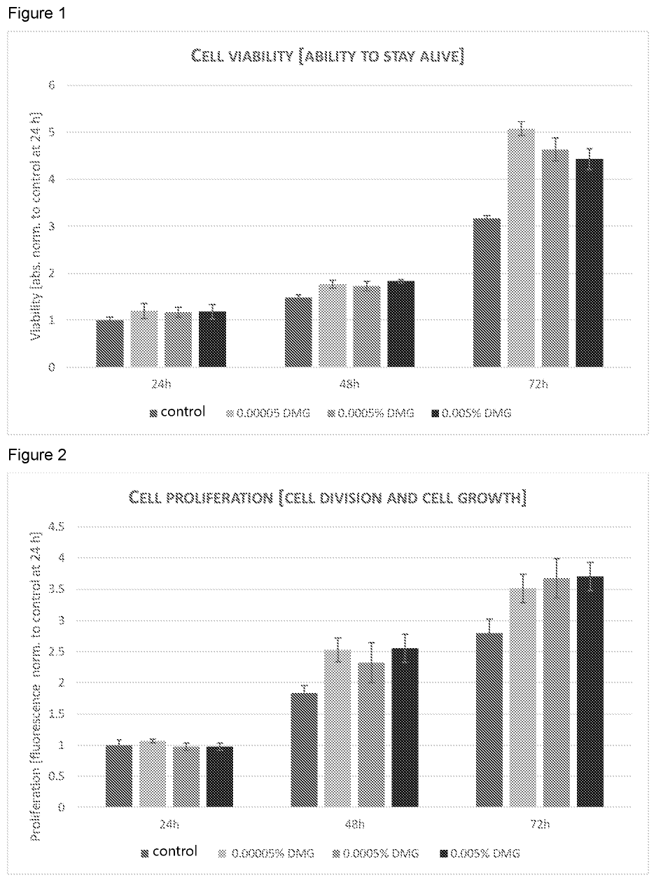

FIG. 1 shows the cell viability after 24 h, 48 h and 72 h of cultivation and a clear increase due to the addition of DMG after 48 h and 72 h can be seen. Cell viability was determined using the MTT assay. The absorption was measured in quadruplicate and normalized to the control at 24 hours. Shown is the mean value with standard deviation.

Evidence of Proliferation:

A so-called CyQUANT assay was carried out to measure the proliferation. In this fluorescence-based assay, the fluorescent dye used binds to DNA (deoxyribonucleic acid), with the content of cellular DNA being a direct measure of the number of cells in a sample.

Human epidermal keratinocyte cells (HaCaT) were seeded in a 96-well plate at a cell density of 5,000 cells/well and cultured in medium (DMEM with 10% FBS, 1% penicillin-streptomycin, 0.5% fungizone). The next day and after a further 24 h as well as 48 h and 72 h of cultivation at 37° C. and 5% by volume CO₂, the cell culture medium without (control) or with the active ingredient concentrations already present was changed. The measurement was carried out analogously to previously published studies in A. Oláh, B. I. Tóth, I. Borbíró, K. Sugawara, A. G. Szöllõsi, G. Czifra, B. Pál, L. Ambrus, J. Kloepper, E. Camera, The Journal of clinical investigation 2014, 124, 3713-3724.

FIG. 2 shows the cell proliferation after 24 h, 48 h and 72 h of cultivation and a clear increase due to the addition of DMG after 48 h and 72 h can be seen. Cell proliferation was determined using the CyQUANT assay. The fluorescence was measured in triplicate and normalized to the control at 24 hours. Shown is the mean value with standard deviation.

Evidence of Migration:

A so-called wound healing assay was carried out to measure migration, based on previously published studies in T. Kawabata, T. Otsuka, K. Fujita, G. Sakai, R. Matsushima-Nishiwaki, O. Kozawa, H. Tokuda, International Journal of Molecular Medicine 2018, 42, 3149-3156. The principle is based on measuring the migration of cells to a cultivation surface that has not yet been colonized over time. For this purpose, 20,000 cells each were seeded into two adjacent wells or cavities separated by a silicone insert (with standardized width) and in medium (DMEM with 5% FBS, 1% penicillin-streptomycin, 0.5% fungizone) for 48 h at 37° C. and 5% by volume CO₂. The plastic insert was then removed (“creation of the wound”) and the migration of the cells was documented by determining the cultivation surface not colonized with cells over time using images. At the same time, the cell culture medium without (control) or with the active substance concentrations of DMG already present in each case was changed directly after removal of the plastic insert (0 h) and after a further 24 h. The evaluation of the images and determination of the cultivation surface not colonized with cells was carried out using specific software (ImageJ).

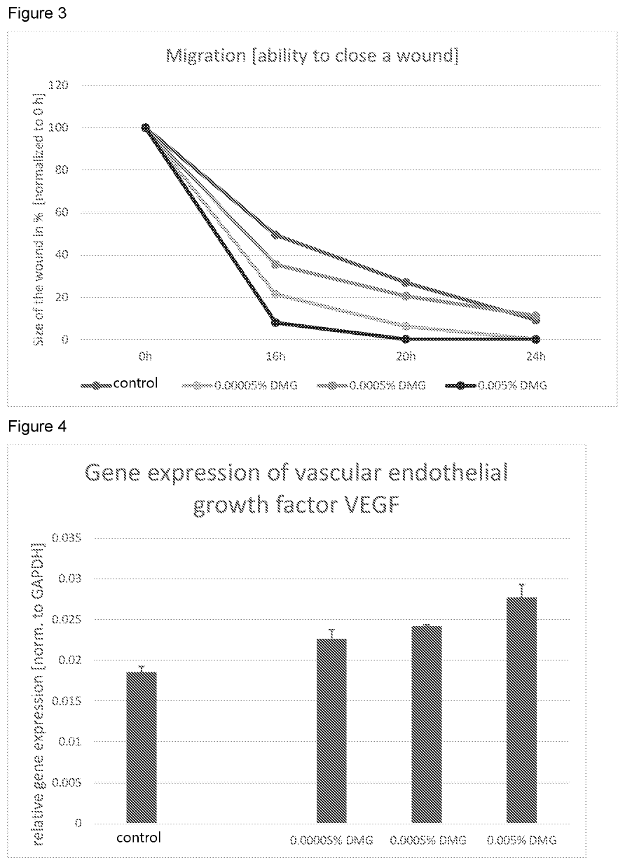

FIG. 3 shows the migration over time and using the different DMG concentrations. Migration or wound closure is significantly faster with DMG compared to controls without DMG. The wound was created at time 0 h and images were taken after 16 h, 20 h and 24 h of cultivation. Based on the image recordings, the cultivation surface, which was not yet colonized with cells, was determined using the ImageJ software. The measured values are normalized to the point in time 0 h (maximum size of the wound) and given in %.

Detection of Gene Expression of VEGF:

VEGF (Vascular Endothelial Growth Factor) promotes the growth and formation of new blood and lymphatic vessels. Gene expression was measured using a standard method known as quantitative real-time PCR (qRT-PCR).

Human epidermal keratinocyte cells (HaCaT) were seeded in a 6-well plate at a cell density of 140,000 cells/well and cultured in medium (DMEM with 10% FBS, 1% penicillin-streptomycin, 0.5% fungizone) at 37° C. and 5% by volume CO₂. The next day, the cell culture medium without (control) or with the active substance concentrations already present in each case was changed, and the cells were harvested after 24 h. Gene expression was measured using qRT-PCR based on studies previously published in B. V. Diaz, M.-C. Lenoir, A. Ladoux, C. Frelin, M. Demarchez, S. Michel, Journal of Biological Chemistry 2000, 275, 642-650.

FIG. 4 shows the gene expression of VEGF after 24 hours of cultivation and a significant increase in gene expression through the addition of DMG can be seen. The relative gene expression determined by qRT-PCR (triple determination, normalized to the respective gene expression of a constitutively expressed gene; GAPDH—glyceraldehyde-3-phosphate dehydrogenase) of VEGF is shown. Shown is the mean value with standard deviation.

Surprisingly, it was found that the parameters relevant for the growth of the HaCaT cells were positively influenced with DMG in the pH range 3.0 to 5.9 and the expression of the growth factor VEGF was significantly increased compared to the treatment of HaCaT cells without DMG.

2. The shampoo according to the invention from Example 1 and the shampoo from Reference Example 1 were also used in an application study on 30 men and 30 women for 6 months. The test subjects were asked to subjectively assess the reduction in their hair loss at the end of the application, with these results being recorded using questionnaires. Significantly fewer hairs fell out per day when the shampoo according to the invention from Example 1 was used.

3. The cream according to the invention from Example 5 and the skin cream from Reference Example 2 were applied to the faces of 20 women (phototype Fitzpatrick I-III, age: 20-64 years) as part of a controlled, blinded half-side test. A quantity of 80 μL (controlled volume measurement via Gilson Pipetman Microman E, M250E, 50-250 microliters, 7-4305) was applied simultaneously to one side of each test person's face and distributed evenly. After one minute (the cream was absorbed into the skin), parameters for the efficacy of the cream were determined subjectively by the test subjects and objectively by the evaluation of a trained and experienced beautician. The evaluated parameters included, for example: skin feel, evenness of the skin, skin moisture.

It has been shown that the test subjects themselves experienced a much more cared-for and softer skin feeling through the application of the cream according to the invention compared to the reference example. At the same time, the complexion of the skin was evaluated objectively as much more even after application of the test cream compared to the reference example.

1.-11. (canceled)

12. A topical composition, comprising:
dimethylglycine and/or a salt of dimethylglycine, wherein the composition has a pH in the range of 3.0 to 5.9.

13. The composition according to claim 12, wherein the composition comprises 0.00001% to 25.0% by weight dimethylglycine and/or a salt of dimethylglycine.

14. The composition according to claim 12, wherein the composition comprises surfactants in an amount of 2% to 40% by weight.

15. The composition according to claim 12, wherein the composition has a viscosity of 800 to 6000 mPa·s.

16. The composition according to claim 12, wherein the composition comprises at least one other active ingredient selected from caffeine, menthol, biotin, zinc PCA, niacinamide, panthenol, ectoin, ubiquinone-10; taurine, pantolactone, echinacea, tocopheryl acetate, and combinations thereof.

17. The composition according to claim 12, wherein the composition comprises at least one additive selected from hair conditioning agents, lipid replenishers, preservatives, stabilizers, fragrances, antioxidants, rheology modifiers, thickeners, conditioning agents, dyes, pearlescent agents, lightening agents, solvents, and combinations thereof.

18. The composition according to claim 12, wherein the composition is a care composition able to promote metabolism of skin and hair.

19. A method for treating hair, scalp, and/or skin of a subject comprising administering a composition comprising:
dimethylglycine and/or a salt of dimethylglycine, wherein the composition has a pH in the range of 3.0 to 5.9.

20. The method of claim 19, wherein the composition comprises 0.00001% to 25.0% by weight dimethylglycine and/or a salt of dimethylglycine, and comprises surfactants in an amount of 2% to 40% by weight.

21. The method of claim 19, wherein the composition has a viscosity of 800 to 6000 mPa·s.

22. The method of claim 19, wherein treating the hair, scalp, and/or skin of the subject comprises treating and/or preventing hair loss.

23. The method of claim 22, wherein the hair loss is selected from the group consisting of alopecia areata (circular hair loss), alopecia androgenetica (hereditary hair loss) in women and men, diffuse alopecia (diffuse hair loss), age-related hair loss (senescent alopecia), and hair loss caused by chemotherapy.

24. The method of claim 22 comprising topically administering the composition to the hair or scalp of the subject.

25. The method of claim 19, wherein treating the hair, scalp, and/or skin of the subject comprises treating and/or improving the appearance of the skin of the subject.

26. The method of claim 25 comprising improving epidermal barrier functions and epidermal barrier integrity of the skin of the subject.

27. The method of claim 25 comprising increasing the moisture content of the skin.

28. The method of claim 25 comprising improving healing of a wound in the skin.

29. The method of claim 25 comprising topically administering the composition to the skin of the subject.

30. The method of claim 19, wherein treating the hair, scalp, and/or skin of the subject is a cosmetic or medicinal treatment.
